(19) United States
(12) Patent Application Publication
(10) Pub. No.: US 2015/0352254 A1
(43) Pub. Date: Dec. 10, 2015

(54) Extracellular Matrix Prostheses for Treating Damaged Biological Tissue

(71) Applicant: CorMartix Cardiovascular, Inc., Roswell, GA (US)

(72) Inventor: Robert G Matheny, Norcross, GA (US)

(21) Appl. No.: 14/832,109

(22) Filed: Aug. 21, 2015

Related U.S. Application Data
(63) Continuation-in-part of application No. 14/566,359, filed on Dec. 10, 2014.
Application No. 14/566,359 is a continuation-in-part of application No. 14/337,915, filed on Jul. 22, 2014.
Application No. 14/337,915 is a continuation-in-part of application No. 14/031,520, filed on Sep. 19, 2013.
Application No. 14/031,520 is a continuation-in-part of application No. 14/031,423, filed on Sep. 19, 2013.

(51) Int. Cl. A61L 27/36; A61L 27/18; A61L 27/38; A61L 27/34; A61L 27/54; A61L 27/58

(52) U.S. Cl. CPC A61L 27/3633; A61L 27/54; A61L 27/58; A61L 27/3839; A61L 27/3834; A61L 27/3804; A61L 27/3629; A61L 27/3625; A61L 27/34; A61L 27/18; A61L 2430/20; A61L 2400/18; A61L 2300/64; A61L 2300/606; A61L 2300/604; A61L 2300/414; A61L 2300/412

(57) ABSTRACT

Particulate structures comprising a particulate extracellular matrix (ECM) component encased in a biomaterial composition, which, when administered to damaged cardiovascular tissue, induce modulated healing.

### CROSS-REFERENCES TO RELATED APPLICATIONS

This application is a continuation-in-part of U.S. application Ser. No. 14/566,359, filed on Dec. 10, 2014, which is a continuation-in-part of U.S. application Ser. No. 14/337,915, filed on Jul. 22, 2014, which is a continuation-in-part of U.S. application Ser. No. 14/031,520, filed on Sep. 19, 2013, which is a continuation-in-part of U.S. application Ser. No. 14/031,423, filed on Sep. 19, 2013, which claims the benefit of U.S. Application No. 61/710,992, filed on Oct. 8, 2012.

### FIELD OF THE INVENTION

The present invention relates to implantable biological prostheses for treating biological tissue. More particularly, the present invention relates to non-antigenic, resilient, biocompatible tissue prostheses and particulate structures that can be used to treat, augment, or replace damaged or diseased biological tissue.

### BACKGROUND OF THE INVENTION

As is well known in the art, tissue prostheses (or grafts) are often employed to treat or replace damaged or diseased biological tissue. However, despite the growing sophistication of medical technology, the use of grafts to treat or replace damaged biological tissue remains a frequent and serious problem in health care. The problem is often associated with the materials employed to construct the grafts.

As is also well known in the art, the optimal graft material should be chemically inert, non-carcinogenic, capable of resisting mechanical stress, capable of being fabricated in the form required, and sterilizable. Further, the material should be resistant to physical modification by tissue fluids, and not excite an inflammatory reaction, induce a state of allergy or hypersensitivity, or, in some cases, promote visceral adhesions. See, e.g., Jenkins, et al., Surgery, vol. 94(2), pp. 392-398 (1983).

Various materials and/or structures have thus been employed to construct grafts that satisfy the aforementioned optimal characteristics, including tantalum gauze, stainless mesh, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastic®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®).

As discussed in detail below, although some of the noted graft materials satisfy some of the aforementioned optimal characteristics, few, if any, satisfy all of the optimal characteristics.

The major advantages of metallic meshes, e.g., stainless steel meshes, are that they are inert, resistant to infection and can stimulate fibroplasia. Several additional disadvantages are fragmentation, which can, and in many instances will, occur after the first year of administration, and the lack of malleability.

Synthetic meshes have the advantage of being easily molded and, except for nylon, retain their tensile strength in or on the body. In European Patent No. 91122196.8 a triple-layer vascular prosthesis is disclosed that utilizes non-resorbable synthetic mesh as the center layer. The synthetic textile mesh layer is used as a central frame to which layers of collagenous fibers are added, resulting in the tri-layered prosthetic device.

There are several drawbacks and disadvantages associated with non-resorbable synthetic mesh. Among the major disadvantages are the lack of inertness, susceptibility to infection, and interference with wound healing.

In contrast to non-resorbable synthetic meshes, absorbable synthetic meshes have the advantage of impermanence at the deployment site, but often have the disadvantage of loss of mechanical strength (as a result of dissolution by the host) prior to adequate cell and tissue ingrowth.

The most widely used graft material for abdominal wall replacement and for reinforcement during hernia repairs is Marlex®, i.e. polypropylene. A major disadvantage associated with polypropylene mesh grafts is that with scar contracture, polypropylene mesh grafts become distorted and separate from surrounding normal tissue.

Gore-Tex®, i.e. polytetrafluoroethylene, is currently believed to be the most chemically inert graft material. However, a major problem associated with the use of polytetrafluoroethylene is that in a contaminated wound it does not allow for any macromolecular drainage, which limits treatment of infections.

Extracellular matrix (ECM) is also often employed to construct tissue prostheses and particulate structures. Illustrative are the grafts disclosed in U.S. Pat. No. 3,562,820 (tubular, sheet and strip grafts formed from submucosa adhered together by use of a binder paste, such as a collagen fiber paste, or by use of an acid or alkaline medium) and U.S. Pat. No. 4,902,508 (a three layer tissue graft composition derived from small intestine comprising tunica submucosa, the muscularis mucosa, and stratum compactum of the tunica mucosa), and the particulate structures disclosed in Co-Pending application Ser. No. 14/566,404.

Although many of the ECM based tissue prostheses or grafts satisfy many of the aforementioned optimal characteristics, when the ECM graft comprises two or more sheets, i.e. a multi-sheet laminate, such as disclosed in Co-pending application Ser. No. 14/031,423, the laminate structure can, and in some instances will, delaminate.

Thus, readily available, versatile vascular grafts that are not prone to calcification, thrombosis, intimal hyperplasia and delamination would fill a substantial and growing clinical need.

It is therefore an object of the present invention to provide tissue prostheses that substantially reduce or eliminate (i) the risk of thrombosis, (ii) intimal hyperplasia after intervention in a vessel, (iii) the harsh biological responses associated with conventional polymeric and metal prostheses, and (iv) the formation of biofilm, inflammation and infection, and (v) delamination.

It is another object of the present invention to provide tissue prostheses and particulate structures that induce modulated healing; particularly, neovascularization, host tissue proliferation, bioremodeling, and regeneration of tissue and associated structures with site-specific structural and functional properties.

It is another object of the present invention to provide tissue prostheses and particulate structures that are capable of administering a pharmacological agent to host tissue and, thereby produce a desired biological and/or therapeutic effect.

### SUMMARY OF THE INVENTION

The present invention is directed to non-antigenic, resilient, bioremodelable, biocompatible tissue prostheses that can be engineered into a variety of shapes and used to repair, augment, or replace mammalian tissues and organs. The present invention is also directed to particulate structures for repairing, augmenting and/or replacing tissues and organs.

As discussed in detail herein, in a preferred embodiment, the tissue prostheses comprise an extracellular matrix (ECM) sheet member comprising an ECM composition having at least one ECM material and at least one defined surface. In a preferred embodiment, the defined surface comprises a biomaterial composition coated surface. In a preferred embodiment, the biomaterial composition comprises an ECM-mimicking biomaterial composition comprising poly(glycerol sebacate) (PGS).

In some embodiments of the invention, the tissue prostheses comprise a multi-sheet laminate structure. In some embodiments, the multi-sheet laminate structure comprises a base ECM sheet member comprising an ECM composition comprising an ECM material and a top ECM-mimicking biomaterial composition coated surface that is in communication with (i.e. in contact with) a non-coated surface of a second ECM sheet member.

In some embodiments of the invention, the particulate structures comprise a particulate component comprising an ECM composition having at least one ECM material that is encased in a biomaterial composition.

In some embodiments of the invention, the biomaterial composition comprises an ECM-mimicking biomaterial composition comprising poly(glycerol sebacate) (PGS).

In a preferred embodiment of the invention, the ECM materials comprise a decellularized ECM material from a mammalian tissue source. According to the invention, the mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

In some embodiments of the invention, the ECM composition and/or biomaterial composition and, hence, tissue prostheses and particulate structures formed therewith further comprise at least one additional biologically active agent or composition, i.e. an agent that induces or modulates a physiological or biological process, or cellular activity, e.g., induces proliferation, and/or growth and/or regeneration of tissue.

In some embodiments, the biologically active agent comprises a cell, such as a human embryonic stem cell, fetal cardiomyocyte, myofibroblast, mesenchymal stem cell, etc.

In some embodiments, the biologically active agent comprises a growth factor, such as a transforming growth factor-alpha (TGF-α), transforming growth factor-beta (TGF-β), fibroblast growth factor-2 (FGF-2), basic fibroblast growth factor (bFGF), and vascular epithelial growth factor (VEGF).

In some embodiments, the ECM composition and/or biomaterial composition and, hence, tissue prostheses and particulate structures formed therewith further comprises at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include, without limitation, antibiotics, anti-viral agents, analgesics and anti-inflammatories.

In some embodiments of the invention, the pharmacological agent comprises a statin, i.e. a HMG-CoA reductase inhibitor, such as cerivastatin.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of an ECM member, in accordance with the invention;

FIG. 2 is front plan view of the ECM member shown in FIG. 1, in accordance with the invention;

FIG. 3A is a front plan view of one embodiment of a multi-sheet or layer pre-laminate structure, in accordance with the invention;

FIG. 3B is a front plan view of one embodiment of a multi-sheet tissue prosthesis formed from the pre-laminate structure shown in FIG. 3A, in accordance with the invention;

FIG. 4 is perspective view of another embodiment of a multi-sheet laminate tissue prosthesis, in accordance with the invention;

FIG. 5 is a side or edge plan view of the tissue prosthesis shown in FIG. 4, in accordance with the invention;

FIG. 6 is perspective view of another embodiment of a multi-sheet laminate tissue prosthesis, in accordance with the invention;

FIG. 7 is a side or edge plan view of the tissue prosthesis shown in FIG. 6, in accordance with the invention; and

FIG. 8 is a front sectional view of one embodiment of a particulate structure, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra; are hereby incorporated by reference in their entirety.

As used in this specification and the appended claims, the singular forms “a, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### DEFINITIONS

The terms “graft” and “endograft” are used interchangeably herein, and mean and include a structure that is configured for implantation in a cardiovascular structure, e.g., a cardiovascular vessel.

The term “particulate”, as used herein, means and includes a structure having a mean particle size in the range of 20-2000 microns.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “ECM-mimicking biomaterial”, and “ECM-mimicking material” are used interchangeably herein, and mean and include a biocompatible and biodegradable biomaterial that induces neovascularization and bioremodeling of tissue in vivo, i.e. when disposed proximate damaged biological tissue. The term “ECM-mimicking” thus includes, without limitation, ECM-mimicking polymeric biomaterial compositions; specifically, poly(glycerol sebacate) (PGS).

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGS), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or anti-thrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antiobiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “therapeutically effective”, as used herein, means that the amount of the “pharmacological agent” and/or “biologically active agent” and/or “pharmacological composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The term “adolescent”, as used herein, means and includes a mammal that is preferably less than three (3) years of age.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

The present invention is directed to non-antigenic, resilient, bioremodelable, biocompatible tissue prostheses that can be engineered into a variety of shapes and used to repair, augment, or replace mammalian tissues and organs.

The present invention is also directed to particulate structures for repairing, augmenting and/or replacing tissues and organs.

As stated above, in some embodiments, the tissue prostheses comprise an ECM sheet member having at least one defined surface. In a preferred embodiment, the defined surface comprises a biomaterial composition coated surface.

In some embodiments, the tissue prostheses comprise a multi-sheet laminate structure. In some embodiments, the multi-sheet laminate structure comprises a base ECM sheet member comprising an ECM material and a top biomaterial composition coated surface that is in communication with (i.e. in contact with) a non-coated surface of a second ECM sheet member.

In some embodiments, the multi-sheet laminate structure comprises a plurality of base ECM sheet members having a top biomaterial composition coated surface and a bottom surface, the bottom surface of each adjoining base ECM sheet member being in communication with a biomaterial composition coated surface of a base ECM sheet member, and a top ECM sheet layer having top and bottom surfaces, the bottom surface of which being in communication with a biomaterial composition coated surface of a base ECM sheet member.

In a preferred embodiment of the invention, the biomaterial composition comprises an ECM-mimicking biomaterial composition. Preferably, the ECM-mimicking biomaterial composition comprises poly(glycerol sebacate) (PGS).

In some embodiments of the invention, the particulate structures comprise a particulate component; preferably, a particulate ECM component comprising an ECM composition having at least one ECM material that is encased in a biomaterial composition.

In some embodiments of the invention, the biomaterial composition similarly comprises an ECM-mimicking biomaterial composition comprising poly(glycerol sebacate) (PGS).

Applicant has found that PGS substantially reduces or eliminates dilation and/or delamination of the prosthesis structures, i.e. between ECM sheet members.

PGS also exhibits numerous beneficial biochemical actions or activities. The properties and beneficial actions resulting therefrom are discussed in detail below.

### PGS Physical Properties

PGS is a condensate of the non-immunogenic compositions glycerol (a simple sugar alcohol) and sebacic acid (a naturally occurring dicarboxylic acid), wherein, glycerol and sebacic acid are readily metabolized when proximate mammalian tissue. The non-immunogenic properties substantially limit the acute inflammatory responses typically associated with other “biocompatible” polymers, such as ePTFE (polytetrafluoroethylene), that are detrimental to bioremodeling and tissue regeneration.

The mechanical properties of PGS are substantially similar to that of biological tissue. Indeed, the value of the Young's modulus of PGS is between that of a ligament (in KPa range) and tendon (in GPa range). The strain to failure of PGS is also similar to that of arteries and veins (i.e. over 260% elongation).

The tensile strength of the PGS is at least 0.28±0.004 MPa. The Young's modulus and elongation are at least 0.122±0.0003 and at least 237.8±0.64%, respectively. For applications requiring stronger mechanical properties and a slower biodegradation rate, PGS can be blended with poly(ε-caprolactone) PCL, i.e. a biodegradable elastomer.

### ECM Mimicking Properties/Actions

It has been established that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

In some embodiments, the ECM-mimicking biomaterial composition further comprises one of the aforementioned ECM materials.

In some embodiments of the invention, the ECM-mimicking biomaterial composition comprises PGS and poly(ε-caprolactone) (PCL). According to the invention, the addition of PCL to the ECM-mimicking biomaterial composition enhances the structural integrity and modulates the degradation of the composition.

In some embodiments, the ECM-mimicking biomaterial composition comprises poly(glycerol sebacate) acrylate (PGSA), which, according to the invention, can be crosslinked and/or cured via the combination of a photoinitiator and radiation.

According to the invention, suitable photoinitiators for radiation induced crosslinking comprise, without limitation, 2-hydroxy-1-[4-hydroxyethoxy)phenyl]-2-methyl-1-propanone (D 2959, Ciba Geigy), 2,2-dimethoxy-2-phenylacetophenone, titanocenes, fluorinated diaryltitanocenes, iron arene complexes, manganese decacarbonyl, methylcyclopentadienyl manganese tricarbonyl and any organometallatic photoinitiator that produces free radicals or cations.

According to the invention, suitable radiation wavelengths for crosslinking and/or curing the ECM-mimicking biomaterial composition comprise, without limitation, visible light; particularly, radiation in the range of approximately 380-750 nm, and ultraviolet (UV) light, particularly, radiation in the range of 10-400 nm, which includes extreme UV (10-121 nm), vacuum UV (10-200 nm), hydrogen lyman α-UV (121-122 nm), Far UV (122-200 nm), Middle UV (200-300 nm), Near UV (300-400 nm), UV-C (100-280 nm), UV-B (280-315 nm) and UV-A (315-400 nm) species of UV light.

In some embodiments, the ECM-mimicking biomaterial composition comprises a co-polymer of PGSA and polyethylene glycol (PEG) diacrylate.

Preferably, the ratio of PGSA to PEG diacrylate used when developing the photocured PGSA is proportional to the physical strength of the biomaterial composition, wherein a ratio of PGSA to PEG diacrylate in the range of 95:05-50:50 comprises a Young's modulus in the range of approximately 0.5-20 MPa respectively.

### ECM Materials

In a preferred embodiment of the invention, the ECM material(s) comprise a decellularized ECM material from a mammalian tissue source. According to the invention, the mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, omentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof.

The ECM material can also comprise collagen from mammalian sources.

In a preferred embodiment, the mammalian tissue source comprises an adolescent mammalian tissue source, i.e. an adolescent mammal, such as a piglet, which is preferably less than three (3) years of age.

In a preferred embodiment, the ECM material is decellularized and, hence, remodelable. According to the invention, the ECM material can be decellularized by various conventional means. In a preferred embodiment, the ECM material is decellularized via one of the unique Novasterilis processes disclosed in U.S. Pat. No. 7,108,832 and U.S. patent application Ser. No. 13/480,204; which are incorporated by reference herein in their entirety.

According to the invention, the ECM material can be formed into a particulate to form a particulate ECM component of the invention and fluidized, as described in U.S. Pat. Nos. 5,275,826, 6,579,538 and 6,933,326, to form an ECM composition of the invention.

According to the invention, various conventional means can be employed to form a particulate ECM material and, hence, component. In some embodiments, the ECM material is formed into a sheet, fluidized (or hydrated), if necessary, frozen and ground.

In some embodiments of the invention, the ground ECM material is subsequently filtered to achieve a desired particulate size. Thus, in some embodiments, the ECM material has a particulate size no greater than 2000 microns. In some embodiments, the ECM material preferably has a particulate size no greater than 500 microns. In a preferred embodiment, the ECM material has a particulate size in the range of about 20 microns to about 300 microns.

In a preferred embodiment of the invention, a plurality of the particulate structures is employed to form biomaterial compositions of the invention. According to the invention, the biomaterial compositions can comprise mixed liquids, mixed emulsions, mixed gels, mixed pastes, or mixed solid particulates. The liquid or semi-solid components of the biomaterial compositions can also comprise various concentrations.

In some embodiments of the invention, the biomaterial compositions thus include a suitable buffer solution, such as saline.

Preferably, the concentration of the liquid or semi-solid components of the biomaterial compositions is in the range of about 0.001 mg/ml to about 200 mg/ml. Suitable concentration ranges thus include, without limitation: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml and about 40 mg/ml. to about 42 mg/ml.

The noted concentration ranges are, however, merely exemplary and not intended to be exhaustive or limiting. It is understood that any value within any of the listed ranges is deemed a reasonable and useful value for a concentration of a liquid or semi-solid component of a biomaterial composition of the invention.

As stated above, in some embodiments of the invention, the ECM composition and/or biomaterial composition and, hence, tissue prostheses and/or particulate structures formed therefrom or therewith further comprise at least one additional biologically active agent or composition, i.e. an agent that induces or modulates a physiological or biological process, or cellular activity, e.g., induces proliferation, and/or growth and/or regeneration of tissue.

In a preferred embodiment of the invention, the biologically active agent is similarly derived from an adolescent mammal, i.e. a mammal less than three (3) years of age.

Suitable biologically active agents include any of the aforementioned biologically active agents, including, without limitation, the aforementioned cells and proteins.

In some embodiments of the invention, the biologically active agent comprises a growth factor selected from the group comprising transforming growth factor-alpha (TGF-α), transforming growth factor-beta (TGF-β), fibroblast growth factor-2 (FGF-2), basic fibroblast growth factor (bFGF) and vascular epithelial growth factor (VEGF).

In some embodiments of the invention, the biologically active agent comprises a protein selected from the group comprising proteoglycans, glycosaminoglycans (GAGs), glycoproteins, heparins, chondroitin sulfate B (dermatan sulfate), chondroitin sulfate A, heparin sulfates, and hyaluronic acids.

In some embodiments of the invention, the protein comprises a cytokine selected from the group comprising a stem cell factor (SCF), stromal cell-derived factor-1 (SDF-1), granulocyte macrophage colony-stimulating factor (GM-CSF), interferon gamma (IFN-gamma), interleukin-3, interleukin-4, interleukin-8, interleukin-10, interleukin-13, leukemia inhibitory factor (LIF), amphiregulin, thrombospondin 1, thrombospondin 2, thrombospondin 3, thrombospondin 4, thrombospondin 5, and angiotensin converting enzyme (ACE).

In some embodiments, the ECM composition and/or biomaterial composition and, hence, tissue prostheses and/or particulate structures formed therefrom or therewith further comprise at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or anti-thrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises one of the aforementioned anti-inflammatory agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. Among the beneficial biochemical actions, Applicant has found that when a statin is added to ECM (wherein a statin augmented ECM member is formed) and the statin augmented ECM member is administered to damaged tissue, the statin interacts with the cells recruited by the ECM, wherein the statin augmented ECM member modulates inflammation of the damaged tissue by modulating several significant inflammatory processes, including restricting expression of monocyte chemoattractant protein-1 (MCP-1) and chemokine (C-C) motif ligand 2 (CCR2).

Further beneficial actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

In some embodiments of the invention, the ECM member and, hence, tissue prosthesis formed therefrom further comprises at least one anchoring mechanism, such as disclosed in Co-pending application Ser. Nos. 13/782,024 and 13/686,131; which are incorporated by reference herein in their entirety.

According to the invention, upon disposing a tissue prosthesis and/or particulate structure of the invention proximate damaged or diseased biological tissue, “modulated healing” is effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the tissue prosthesis and/or particulate. structure is specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase.

In some embodiments, “modulated healing” refers to the ability of a tissue prosthesis and/or particulate structure to alter a substantial inflammatory phase (e.g., platelet or fibrin deposition) at the beginning of the tissue healing process. As used herein, the phrase “alter a substantial inflammatory phase” refers to the ability of a tissue prosthesis and/or particulate structure to substantially reduce the inflammatory response at an injury site.

In such an instance, a minor amount of inflammation may ensue in response to tissue injury, but this level of inflammation response, e.g., platelet and/or fibrin deposition, is substantially reduced when compared to inflammation that takes place in the absence of a tissue prosthesis of the invention.

In some embodiments of the invention, “modulated healing” refers to the ability of a tissue prosthesis and/or particulate structure of the invention to induce host cell and/or tissue proliferation, bioremodeling, including neovascularization, e.g., vasculogenesis, angiogenesis, and intussusception, and regeneration of tissue structures with site-specific structural and functional properties when disposed proximate damaged tissue.

In some embodiments, the term “modulated healing” thus means and includes the ability of tissue prosthesis and/or particulate structure to modulate inflammation and/or induce host cell and/or tissue proliferation and bioremodeling when disposed proximate damaged tissue.

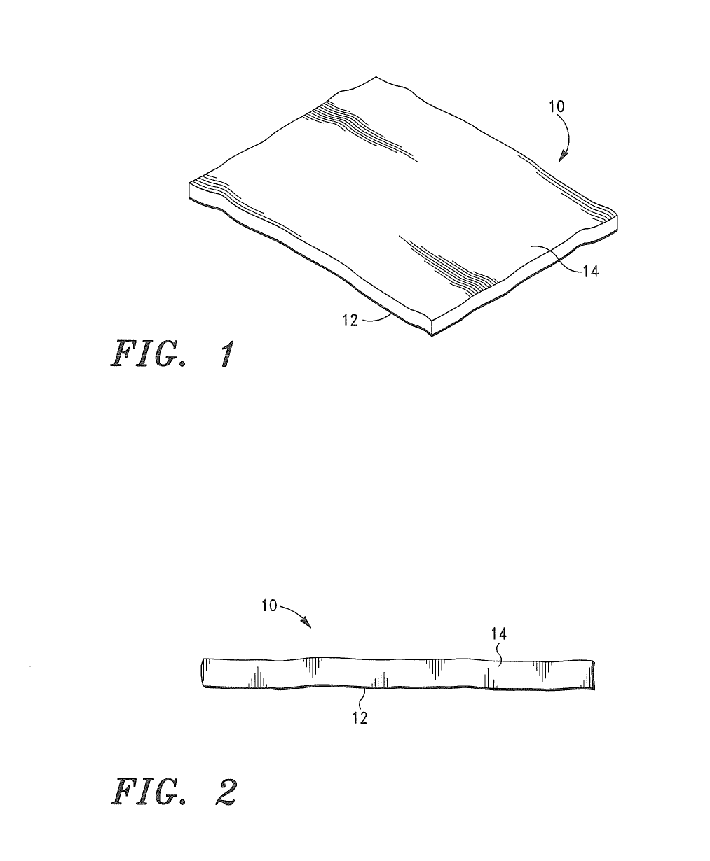

Referring now to FIGS. 1 and 2, there is shown one embodiment of an ECM sheet member of the invention. As illustrated in FIG. 2, the ECM sheet member 10 comprises a biomaterial composition coated surface 14 and a bottom surface 12.

According to the invention, the ECM sheet member 10 can further comprise top and bottom biomaterial composition coated surfaces.

As indicated above, in a preferred embodiment of the invention, the ECM sheet member 10 comprises a decellularized ECM material. As also indicated above, preferably, the ECM material is derived from an adolescent mammal, i.e. a mammal less than three (3) years of age.

According to the invention, the ECM sheet member 10, and, hence tissue prosthesis formed therefrom can comprise various shapes and dimensions to accommodate various applications.

In some embodiments of the invention, the ECM sheet member 10 (and, hence, ECM material thereof) and/or biomaterial composition coated surface 14 further comprises at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells, growth factors and proteins.

In some embodiments, the ECM member 10 (and, hence, ECM material thereof) biomaterial composition coated surface 14 further comprises at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, and anti-inflammatories.

In some embodiments of the invention, the pharmacological agent comprises a statin.

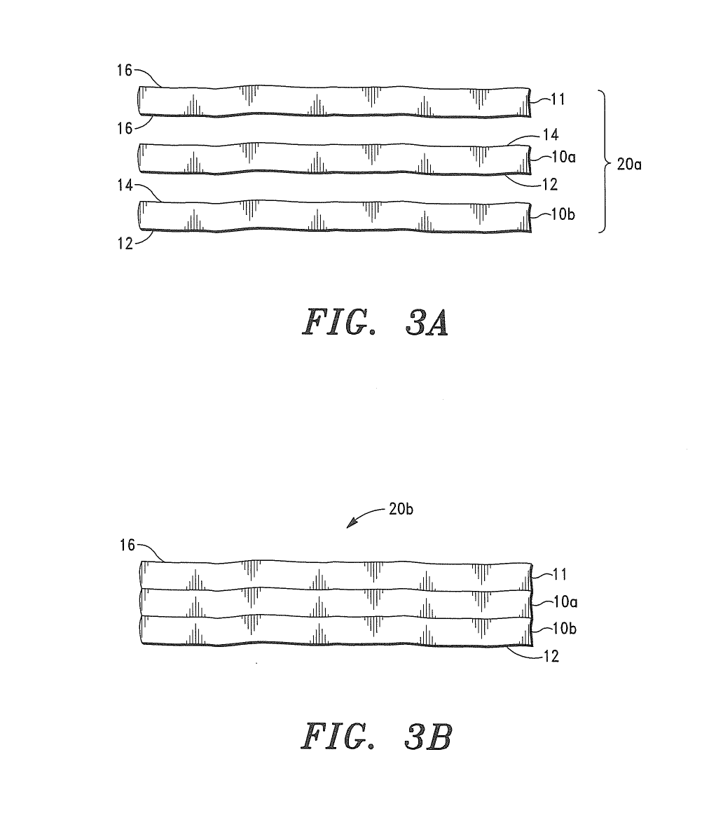

Referring now to FIG. 3A, there is shown a multi-sheet pre-laminate structure 20a that can be employed to construct a multi-sheet tissue prosthesis of the invention. In the illustrated embodiment, the pre-laminate structure 20a comprises first and second ECM sheet members 10a, 10b, each sheet member 10a, 10b having a top biomaterial composition coated surface 14 and a bottom surface 12. The pre-laminate structure 20a further comprises a third ECM sheet member 11 having top and bottom surfaces 16.

Referring now to FIG. 3B, there is shown one embodiment of a multi-sheet tissue prosthesis 20b that is formed from the pre-laminate structure shown in FIG. 3A. As illustrated in FIG. 3B, the bottom surface 12 of the first ECM sheet member 10a is in communication with a biomaterial composition coated surface 14 of the adjoining second ECM sheet member 10b, and the bottom surface 16 of the third ECM sheet member 11 is in communication with the biomaterial composition coated surface 14 of the first ECM sheet member 10a. The resultant structure thus comprises three layer laminated ECM structure with a non-coated top and bottom surface.

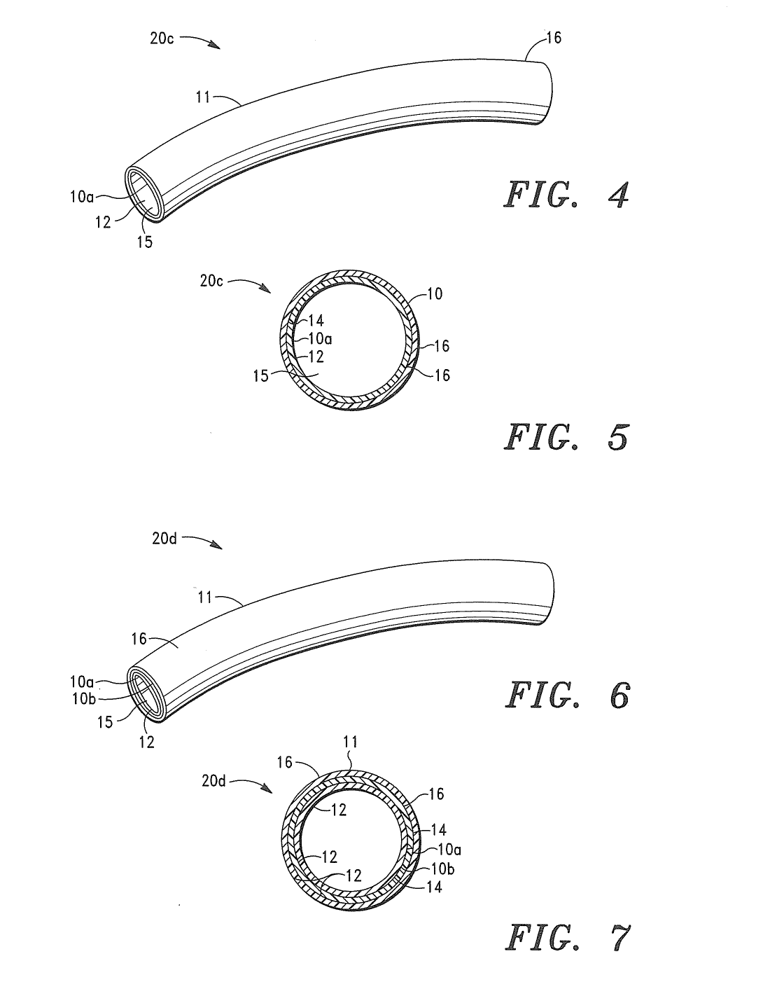

Referring now to FIGS. 4 and 5, there is shown another embodiment of a tissue prosthesis of the invention. As illustrated in FIG. 4, the prosthesis 20c comprises tubular member having a lumen 15 that extends therethrough.

As illustrated in FIG. 5, the prosthesis 20c comprises a bottom or base ECM sheet member 10a that similarly includes a top biomaterial composition coated surface 14 and a bottom surface 12, and an adjoining top ECM sheet member 11 having top and bottom surfaces 16. As further illustrated in FIG. 5, the bottom surface 16 of the ECM sheet member 11 is in communication with the top biomaterial composition coated surface 14 of the base ECM sheet member 10a.

Referring now to FIGS. 6 and 7, there is shown another embodiment of a tissue prosthesis of the invention. As illustrated in FIG. 6, the prosthesis 20d similarly comprises a tubular member having a lumen 15 that extends therethrough.

As illustrated in FIG. 7, the prosthesis 20d comprises first and second ECM sheet members 10a, 10b having biomaterial composition coated surfaces 14 and bottom surfaces 12; the bottom surface 12 of the first ECM member 10a being in communication with the biomaterial composition coated surface 14 of the second adjoining ECM sheet member 10b (or sheet layer), and a top ECM sheet member 11 having top and bottom surfaces 16. As further illustrated in FIG. 7, the bottom surface 16 of the ECM sheet member 11 is in communication with the top biomaterial composition coated surface 14 of the first ECM sheet member 10a.

According to the invention, the multi-sheet tissue prostheses of the invention, including prostheses 20b, 20c and 20d described above, can be formed in any ECM sheet member order; provided, that the surfaces of the top and bottom ECM sheet members are preferably non-coated surfaces.

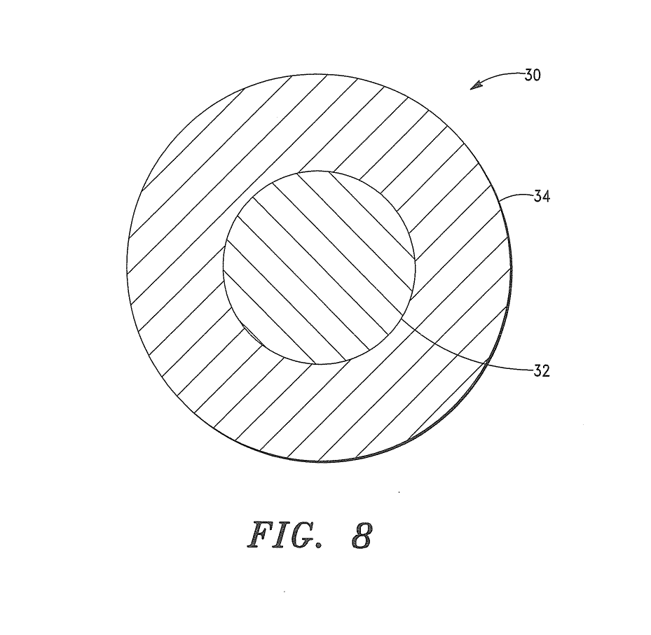

Referring now to FIG. 8, there is shown one embodiment of a particulate structure of the invention. As illustrated in FIG. 8, the particulate structure 30 comprises a particulate component 32 comprising a first composition that is encased in a second composition 34.

As indicated above, according to the invention, the first composition of the particulate component 32 can comprise one of the aforementioned ECM compositions and the second composition 34 can comprise one of the aforementioned biomaterial compositions.

The first composition can also comprise a biomaterial composition and the second composition 34 can comprise an ECM composition.

In a preferred embodiment of the invention, a plurality of the particulate structures shown in FIG. 1 are employed to form a biomaterial composition for treating damaged tissue.

In some embodiments, the biomaterial composition thus comprises a plurality of particulate structures comprising a particulate ECM component comprising an ECM composition, the ECM composition comprising at least one ECM material, each of said plurality of particulate ECM components being encased in a biomaterial composition, the biomaterial composition being configured to induce modulated healing when delivered to damaged biological tissue.

As will readily be appreciated by one having ordinary skill in the art, the present invention provides numerous advantages compared to prior art prosthetic valves. Among the advantages are the following:
The provision of tissue prostheses that substantially reduce or eliminate (i) the risk of thrombosis, (ii) intimal hyperplasia after intervention in a vessel, (iii) the harsh biological responses associated with conventional polymeric and metal prostheses, (iv) the formation of biofilm, inflammation and infection and (v) delamination.
The provision of tissue prostheses and particulate structures that can be effectively employed to treat, reconstruct, replace and improve biological functions or promote the growth of new cardiovascular tissue in a cardiovascular structure.
The provision of tissue prostheses and particulate structures that induce host tissue proliferation, bioremodeling and regeneration of new tissue and tissue structures with site-specific structural and functional properties.
The provision of tissue prostheses and particulate structures that are capable of administering a pharmacological agent to host tissue and, thereby produce a desired biological and/or therapeutic effect.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A biomaterial composition for treating damaged cardiovascular tissue, comprising:
a plurality of particulate structures comprising a particulate extracellular matrix (ECM) component comprising an ECM composition, said ECM composition comprising at least one acellular ECM material, each of said plurality of particulate ECM components being encased in a biomaterial composition comprising poly(glycerol sebacate) (PGS), said biomaterial composition being configured to induce modulated healing when delivered to damaged biological tissue.

2. The biomaterial composition of claim 1, wherein said acellular ECM material comprises mammalian tissue selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, omentum extracellular matrix, heart extracellular matrix and lung extracellular matrix.

3. The biomaterial composition of claim 2, wherein said first acellular ECM material comprises adolescent ECM material.

4. The biomaterial composition of claim 1, wherein said first ECM composition further comprises at least one exogenously added first biologically active agent.

5. The biomaterial composition of claim 4, wherein said first biologically active agent comprises a cell selected from the group consisting of a human embryonic stem cell, fetal cardiomyocyte, myofibroblast, and mesenchymal stem cell.

6. The biomaterial composition of claim 4, wherein said first biologically active agent comprises a growth factor selected from the group consisting of a transforming growth factor-alpha (TGF-α), transforming growth factor-beta (TGF-β), fibroblast growth factor-2 (FGF-2), basic fibroblast growth factor (bFGF), and vascular epithelial growth factor (VEGF).

7. The biomaterial composition of claim 1, wherein said first ECM composition further comprises at least a pharmacological agent.

8. The biomaterial composition of claim 7, wherein said pharmacological agent comprises an agent selected from the group consisting of an antibiotic, anti-viral agent, analgesic, anti-inflammatory, anti-neoplastic, anti-spasmodic, and anticoagulant and anti-thrombic agent.

9. The biomaterial composition of claim 7, wherein said pharmacological agent comprises a statin selected from the group consisting of atorvastatin, cerivastatin, fluvastatin, lovastatin, mevastatin, pitavastatin, pravastatin, rosuvastatin and simvastatin.

10. A method for treating damaged cardiovascular tissue, comprising:
identifying damaged tissue at a cardiovascular structure site;
providing a biomaterial composition comprising a plurality of particulate structures comprising a particulate extracellular matrix (ECM) component, said particulate ECM component comprising an ECM composition comprising at least one acellular ECM material, each of said plurality of particulate ECM components being encased in a biomaterial composition comprising poly(glycerol sebacate) (PGS);
administering said biomaterial composition to said damaged tissue at said cardiovascular structure site, wherein said biomaterial composition induces modulated healing of said damaged tissue.

11. The method of claim 10, wherein said acellular ECM material comprises mammalian tissue selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, omentum extracellular matrix, heart extracellular matrix and lung extracellular matrix.

12. The method of claim 11, wherein said first acellular ECM material comprises adolescent ECM material.

13. The method of claim 10, wherein said first ECM composition further comprises at least one exogenously added first biologically active agent.

14. The method of claim 13, wherein said first biologically active agent comprises a cell selected from the group consisting of a human embryonic stem cell, fetal cardiomyocyte, myofibroblast, and mesenchymal stem cell.

15. The method of claim 13, wherein said first biologically active agent comprises a growth factor selected from the group consisting of a transforming growth factor-alpha (TGF-α), transforming growth factor-beta (TGF-β), fibroblast growth factor-2 (FGF-2), basic fibroblast growth factor (bFGF), and vascular epithelial growth factor (VEGF).

16. The method of claim 10, wherein said first ECM composition further comprises at least a pharmacological agent.

17. The method of claim 16, wherein said pharmacological agent comprises an agent selected from the group consisting of an antibiotic, anti-viral agent, analgesic, anti-inflammatory, anti-neoplastic, anti-spasmodic, and anticoagulant and anti-thrombic agent.

18. The method of claim 16, wherein said pharmacological agent comprises a statin selected from the group consisting of atorvastatin, cerivastatin, fluvastatin, lovastatin, mevastatin, pitavastatin, pravastatin, rosuvastatin and simvastatin.
